(19) United States
(12) Patent Application Publication
(10) Pub. No.: US 2006/0258934 A1
(43) Pub. Date: Nov. 16, 2006

(54) Magnetic resonance locating method

(76) Inventors: Michael Zenge, Essen (DE); Steffen Weiss, Hamburg (DE); Tobias Schaeffter, Hamburg (DE); Ralph Sinkus, Hamburg (DE)

Correspondence Address: PHILIPS INTELLECTUAL PROPERTY & STANDARDS, 595 MINER ROAD, CLEVELAND, OH, 44143, US

(21) Appl. No.: 10/554,307

(22) PCT Filed: Apr. 13, 2004
(86) PCT No.: PCT/IB04/01188

§ 371 Date: Oct. 21, 2005

(30) Foreign Application Priority Data
Apr. 23, 2003 (EP) 03101109.1

(51) Int. Cl. A61B 5/05

(52) U.S. Cl. 600/410

(57) ABSTRACT

The invention relates to a magnetic resonance method for locating interventional devices, in particular in vivo, in which the interventional device bears a marking which in magnetic resonance images influences the measured signals or generates its own measured signals, where the measured signals are processed by means of a one-dimensional signal processing method in order to suppress noise and artefacts. This may in particular be the maximum entropy method, which can be further expanded by the use of model functions. These model functions are subtracted from the measured signals during the iterative method in order in this way to additionally improve the elimination of artefacts. As an alternative to the use of the maximum entropy method, the use of filters, in particular Wiener filters or bandpass filters, is also possible.

The invention relates to a magnetic resonance method for locating interventional devices, in particular in vivo, in which the interventional device bears a marking which in magnetic resonance acquisitions influences the measured signals or generates its own measured signals.

The use of magnetic resonance methods (MR methods) in medical interventions is becoming increasingly important. On the one hand, MR imaging is distinguished by excellent soft tissue contrast and by any orientation of the image planes; on the other hand, a health risk to patients and operating staff on account of ionizing radiation, as used in X-ray methods, is avoided.

Nevertheless, when visualizing and locating interventional devices for insertion into the body of a patient, in particular catheters, there is the problem that said devices cannot usually be observed directly. Whereas in imaging methods based on the use of X-ray radiation even very small metal wires bring about an image contrast sufficient to visualize the catheter, in magnetic resonance imaging these bring about only an insufficient signal reduction, since such small objects displace only a very small volume of water. For this reason, the visibility of the interventional devices must be increased in another way, and various methods have been developed for this purpose.

The locating methods described in the literature are subdivided into two categories. In active methods the interventional device has a receiving coil so that signals can be received from the surroundings of the device via an additional channel. By contrast, passive methods visualize the interventional device in the MR image by the contrast with respect to the surrounding tissue.

In the active method sector, two catheter locating methods have thus far been established. Firstly, it is possible for a small receiving coil to be incorporated in the catheter tip, which receiving coil is connected to a reception channel via a coaxial cable through the catheter (C. L. Dumoulin et al., Magn. Reson. Med. 29, 411-415 (1993)). The great advantage of this method is the possibility, by applying field gradients, of determining the coordinates of the catheter tip from projections in the corresponding spatial directions. Moreover, the method is compatible with all rapid imaging methods and thus has real-time capability.

As an alternative to the use of a receiving coil, it is also possible for an elongated antenna to be inserted into the catheter, which antenna then receives MR signals along the catheter. In this way, even instruments having a small diameter such as guidewires and neurological catheters can be made visible. One particular field of application is in intravascular imaging.

In both methods it is disadvantageous that the line for HF excitation pulses which runs through the catheter to the reception channel can unintentionally act as an antenna. It has thus been shown that a guidewire can heat up to 74° C. after 30 seconds of a gradient-echo sequence. The resonance conditions in this case are varied and in clinical practice are difficult to monitor.

On the other hand there are the passive techniques, in which the visibility of the catheter is increased in a specific way. One possibility is the use of contrast media, with catheters being used either whose volume is filled with an appropriate medium (Gd-DTPA) or whose sheath is coated in a contrast-amplifying manner.

Another approach consists in generating susceptibility artefacts in the MR image by disturbing the static magnetic field B₀. Conventional polyethylene catheters may for this purpose be prepared with paramagnetic rings (Dy₂O₃). A working group at the University Clinic of RWTH Aachen has developed an alternative method in which a local field inhomogeneity is brought about by a wire loop in the catheter, which wire loop is then connected to an external power source (A. Glowinski et al., Magn. Reson. Med. 38, 253-258 (1997)). In this way, the image artefact can be controlled via the source during the intervention.

In these three passive visualization techniques, the positive aspects are that it is possible to make the entire length of the catheter visible and that the methods are compatible with all imaging methods. The disadvantages are that all methods are comparatively time-consuming and the coordinates of the catheter position-are not directly accessible. Automated tracing of the catheter is therefore not possible.

According to another locating method described by M. Burl, Magn. Reson. Med. 36, 491-493 (1996) and S. Weiβ, Proc. ISMRM, 544 (2001), a catheter, also referred to as an OptiMa catheter, is fitted at its tip with an electronically isolated resonant circuit which is tuned to the Larmor frequency. When a B₁ HF pulse is transmitted, the resonant circuit is excited and causes a local increase in resonance of the B₁ field, which locally increases the flip angle and thus the signal. The resonant circuit can be detuned optically by way of a photodiode which is illuminated by a lightguide running through the catheter, and hence the signal amplification can be turned on and off. The signal background is suppressed by subtracting an on/off signal. The measured signals obtained when the marking is activated and deactivated are also referred to as on-projection and off-projection, respectively.

This method is distinguished in that the catheter coordinates are directly accessible and the technique is compatible with all imaging methods. Patient safety is also ensured since a lightguide running through the catheter, unlike an electrical guide, cannot act as an antenna which heats up considerably under the effect of HF pulses. Finally, the method also has real-time capability.

However, one disadvantage in this prior art is that the detection of the interventional device is not ensured in every case, since the determination of the coordinates can be disrupted by noise and artefacts. The position of the device is determined from the difference between on-projection and off-projection by the sampled value with maximum signal amplitude. However, the signal quality is adversely affected by various effects. Firstly, the quality of the signal is highly dependent on the distance between the receiving coil and the marking on the interventional device, since the pulse is weaker the further the receiving coil is from the origin of the signal. Nevertheless, the signal quality is affected to a much greater extent by the orientation of the device with respect to the transmitting and receiving coil. When there are large angles between the resonant coil, locally approximated by a dipole moment, and the field lines of the transmitting and receiving coil, these couple only to a weak extent.

Apart from the high degree of variation in the pulse brought about by the interventional device, the location operation is significantly disrupted by extended artefacts. Frequently, the background signal in the difference is not fully extinguished, and this can be attributed to the fact that the magnetization, at the moment of excitation for the respective projections, is not in the same state but rather is subjected to a transient process. For this reason, the amplitudes in the on-projection and off-projection are at different levels. The artefacts brought about in this way will be referred to herein below as transient artefacts.

Further artefacts, also referred to as image slice artefacts, arise since in each new detection the magnetization in the previous image slice has generally not fully died out. This residual magnetization then dies out between on-projection and off-projection and therefore appears in the difference projection as an artefact in the center of the data vector. Finally, movements caused by breathing and the heartbeat and also pulsed blood flow may have a negative effect on the quality of the signal.

A reliable conclusion about the position of the interventional devices can no longer be drawn if the background, caused by noise and artefacts, of the amplitude of the pulse emanating from the marking of the interventional device gets closer. Based on this prior art, it is therefore an object of the present invention to provide a magnetic resonance method for locating interventional devices, in which noise and artefacts are suppressed to the extent that the detectability of the signal coming from the marking of the interventional device is always ensured.

The object is achieved according to the invention by a magnetic resonance method as claimed in the precharacterizing part of claim 1, in which the measured signals are processed by means of a one-dimensional signal processing method in order to improve the location operation.

Furthermore, the invention also relates to an apparatus and to a computer program for carrying out the method according to the invention.

In the context of this invention, the term interventional device is understood to mean in particular catheters, but also biopsy needles, minimally invasive surgical instruments, guidewires, stents, etc. The marking on the interventional device may in particular be a resonant circuit at the tip of an OptiMa catheter; however, it may also be other types of arrangement such as, for example, a microcoil as used for active locating methods. A marking which can be switched on and off, allowing the separate recording of measured signals in the on and off state, also referred to in the context of this invention as on-projection and off-projection, is advantageous here, so that the position determination of the marking is possible by difference formation between on-projection and off-projection.

The one-dimensional signal processing method is preferably an iterative method as provided for problems which cannot be solved directly by analysis. The so-called maximum entropy method is particularly suitable.

The maximum entropy method (ME method) is an iterative, nonlinear method for signal restoration. The ME method solves underdefined problems by selecting, from all the solutions that are compatible with the data, that solution having the maximum entropy. One particular advantage is given by the possibility of taking into account prior knowledge about the measuring process by including additional parameters in the algorithm.

The initial problem on which the maximum entropy method is based can be described in general terms as follows:

The object is to determine a distribution function as the best estimate for a distribution of states. Usually there are an infinite amount of distributions which are compatible with the secondary conditions. The principle of maximum entropy means that from these, that distribution which has the maximum entropy is to be selected. This choice is the only one that is consistent with the data without adding additional information.

One approach, based on probability theory, for substantiating the ME method is described inter alia by G. J. Daniell and S. F. Gull in IEE Proc. 127, Pt. E, 170-172 (1980). This states that the following is true when the input signal is superposed by white noise:

$${χ}^{2} = {\sum }_{signal} \frac{{( deviation between measured signal and forecast signal )}^{2}}{{( error in measured signal )}^{2}}$$

The probability for the estimated signal is then proportional to exp(−½χ²). The ME method is thus based on a χ² minimization with adaptation of the estimated signals to the measured data. The algorithm which is attributed to the authors Skilling and Bryan, Mon. Not. astr. Soc. 221, 111-124 (1984) and is distinguished by a high convergence rate has proven to be particularly suitable for use in the method according to the invention.

According to an advantageous design of the invention, to suppress artefacts occurring in the measured signals, model functions are formed, adapted and subtracted from the measured signals as the iterative method is carried out. The adaptation of the model functions to the recorded measured signals (the on-projection) expediently takes place by the model, functions being calculated with a scaling parameter. The incorporation into the maximum entropy algorithm can take place in two different ways. The scaling parameter can be adapted anew after each iteration step or just once prior to the ME iteration. In the test carried out for this purpose, in the first case the parameter was determined as a function of noise with an accuracy from 1 to 4%, whereas in the second case the relative deviation was approximately twice as great. On the other hand, in the second case approximately 10% less calculation time was required.

In the case of the artefacts that are to be eliminated, a distinction must be made, as already mentioned above, between transient artefacts and image slice artefacts Since the occurrence of transient artefacts can be attributed to the fact that the magnetization at the time of excitation, for measurements in which the marking on the interventional device is switched on and off, is not in the same state, in particular when using the abovementioned OptiMa catheter which has a marking that can be switched optically, the background signal is not completely extinguished by forming the difference of measurements with activated and deactivated marking.

For this reason, a recorded off-projection can be used as model function to suppress the transient artefacts. By way of the abovementioned scaling parameter, the model function created in this way can be adapted to the recorded measured signals, by the on-projection and off-projection being compared with one another. During the χ² adaptation the model function is then subtracted from the measured signal. The signal defining the position of the interventional device is thus amplified relative to the background, so that the sampled value with the maximum signal amplitude can be assigned to the position with considerably increased certainty.

By contrast, in order to suppress the image slice artefacts which may also occur and which can be attributed to the fact that in the individual detections the magnetization in the previous image slice has generally not completely died out, other model functions must be used. In this case, rectangular or Gaussian functions may be used, which can likewise be adapted by way of a scaling parameter. The reason for the type of model function used can be seen in the considerably narrower image of the image slice artefacts, which are of the order of magnitude of the width of an image slice, compared to transient artefacts.

In order to be able to draw a conclusion about the capability of signal processing relative to the quality of the input signal, two different parameters are used. Firstly, the signal-to-noise ratio S/N provides information about the noise minimization following signal processing, although no account is taken of any signal interference on account of artefacts which under some circumstances impair the determination of the position of the interventional device much more than noise alone. More information is thus provided by the signal-to-interference ratio S/A, which besides the high frequency noise also takes the low frequency artefacts into account. These are the quotients of the useful signal power and the total power reduced by the power of the DC signal. When the noise in a signal is dominant, the S/A strives againist the S/N ratio. The suppression of noise alone however, only leads to a slight improvement in the S/A ratio. The S/A ratio is much more suited than the S/N ratio to assess the certainty with which the position is determined. Thus, in the investigations carried out, it has been found that there is a reliable detectability of the position of the interventional device when an S/A ratio of ≧20 dB is measured.

The convergence rate of the maximum entropy algorithm is primarily dependent on the noise. Independently thereof, the number of iterations can be influenced by a suitable choice of the user-defined background, that is to say of the start value of the iteration, since the success of the χ² adaptation at the start of the iteration varies depending of the choice of this start value. An increase in the convergence rate is particularly important when signal processing in real time is desired.

It has been found that in the method according to the invention, without additional use of model functions, the convergence rate is at a maximum when the mean value of the measured signals is selected as the start value for the iteration. At the same time, the maximum S/N ratio is also obtained for this choice of the user-defined background, whereas the S/A ratio is largely independent of the choice of start value for the iteration. Given a suitable choice of start value, the ME algorithm converges in less than ten iteration steps. If, on the other hand, model functions in accordance with what has been stated above regarding the optimization of the signal processing and elimination of artefacts are used, it has been found to be expedient to use the mean value of the difference between measured signals and model function as start value for the iteration. This mean value is considerably less than the mean value of the measured signal, since the significant artefacts have already been suppressed by the model function.

A further possibility for increasing the quality of the measured signals that is offered by the maximum entropy method consists in suppressing noise and artefacts by extinguishing the corresponding high frequency or low frequency input signal fractions. Since the reliable determination of the position of the interventional device is impaired to a greater extent when there are extended artefacts having a high amplitude than by noise alone, it is particularly important to suppress said artefacts. Both in vitro and in vivo, artefacts which were four to five times wider than the pulse emanating from the marking were usually observed. Given a total number N of 256 sampled values, these are typically artefacts which extend over more than 32 sampled values.

The suppression of an unnecessarily large amount of signal fractions nevertheless leads to losses in the S/N ration, and this can be attributed to the fact that by extinguishing these low frequency signal fractions the mean value is significantly decreased while the noise essentially remains unaffected. Accordingly, for example given an artefact width of 32 sampled values, the S/A ratio is at a maximum when 8 low frequency sampled values are eliminated, and this corresponds to the quotient of the total number of sampled values and the number of sampled values across which one artefact extends. Moreover, the extinguishing of too many low frequency signal fractions which contain a lot of signal power when massive artefacts occur may lead to the convergence criteria for the ME algorithm no longer being fulfilled if too low a start value is used for the iteration.

An improvement in the signal quality by eliminating noise and thus an improvement in the S/N ratio may be obtained by extinguishing high frequency sampled values in the spectrum. The extinguishing of too many high frequency sampled values nevertheless leads to a significant decrease in the useful signal power, which is associated with losses in the S/A ratio. Given a total number of N=256 sampled values, it was found that no more than 96 high frequency sampled values should be extinguished, since in this range the spectrum of the useful signal is negligible. A significant effect on the number of iteration steps by suppressing high frequency or low frequency signal fractions and hence on the calculation time could not be established.

In vivo experiments, it was possible to show that reliable position determination is possible by eliminating signal fractions even when there are input signals that contain a lot of noise and are highly disrupted by artefacts. However, it must be pointed out that in the expanded ME method described above, in which adapted model functions are subtracted from the measured signals, the elimination of sampled values is not useful. This can be attributed to the fact that during the χ² adaptation the artefacts corresponding to the model function are subtracted from the measured signal, with it being necessary for the estimated signal to be brought into correspondence with this difference signal. An additional extinguishing of low frequency signal fractions would therefore lead to a falsification, which no longer permits adaptation.

Besides the iterative methods described above, particularly the maximum entropy method, it is also possible to use other one-dimensional signal processing methods such as, for example, filters. In principle, both filters having a finite impulse response and filters having an infinite impulse response are suitable, these also being referred to by the terms FIR (finite impulse response) and IIR (infinite impulse response). Such filters are known in principle to the person skilled in the art. Two typical filters which have been found to be suitable for achieving the object of the invention are the Wiener filter and the bandpass filter.

The Wiener filter can be depicted in Fourier form as follows:

$$W = \frac{1}{H} * \frac{{Φ}_{ff} {\vert H \vert}^{2}}{{Φ}_{ff} {\vert H \vert}^{2} + {Φ}_{nn}}$$

In this case, H is the transfer function of the measurement system and (Φ_{ff} and Φₙₙ are the power density spectra of the sought-after signal fₖ and noise nₖ.

The Wiener filter is particularly suitable for improving the S/N ratio, that is to say for effectively suppressing noise. Artefacts, on the other hand, are suppressed to a poorer extent than when the maximum entropy method is used.

A further suitable filter is the bandpass filter which has proven to be effective for suppressing noise and artefacts. The certainty with which an interventional device can be located could be considerably increased with the aid of a bandpass filter. The bandpass filter is less suitable only in the case of suppressing narrow artefacts, such as image slice artefacts for example.

The choice of the most suitable signal processing method depends on the exact nature of the problem. On the one hand, the maximum entropy method gives the best results in terms of artefact and noise suppression, particularly when implementing the additional features mentioned above. On the other hand, the ME method, as an iterative method, requires considerably more calculation time than when a filter is used. While said calculation time is in the range from 1 to 2 ms for a filter, for the ME method the calculation time may be >100 ms, depending on the total number of sampled values. Therefore, when there are very strict requirements in terms of the brevity of the calculation time for real-time visualization, a filter should be used instead of the ME method.

A further improvement in the location of an interventional device can be achieved, when there are a number of measured signals being used for locating purposes, in that after processing of the measured signals by means of the one-dimensional signal processing method a check as to coincidence of the positions of the interventional device determined by way of the processed measured signals is carried out. Such a check is provided in particular when using the above-described OptiMa catheter, in which case a number of receiving coils which receive the measured signals in parallel are located on the body of the patient. Although these measured signals differ from one another during the location operation in terms of the amplitude, the same position in terms of space should be obtained for the interventional device.

When checking the processed measured signals with regard to coincidence, after processing of the measured signals a check is then made as to whether the positions determined via the individual receiving coils coincide. Such a full or partial coincidence additionally increases the probability that the determined position is correct.

Preferably, the various measured signals being used to locate the interventional device are processed jointly in the one-dimensional signal processing method, so that the effects on the position determination for the individual measured signals are also the same. This is possible both by using an iterative method such as the maximum entropy method and by using a filter. The determined positions for the interventional device can then be checked with regard to coincidence. The correlation of the measured signals can also be calculated directly by the one-dimensional signal processing method in order in this way to obtain a measure of the coincidence of the signal spectra.

The invention will be further described with reference to examples of embodiments shown in the drawings to which, however, the invention is not restricted.

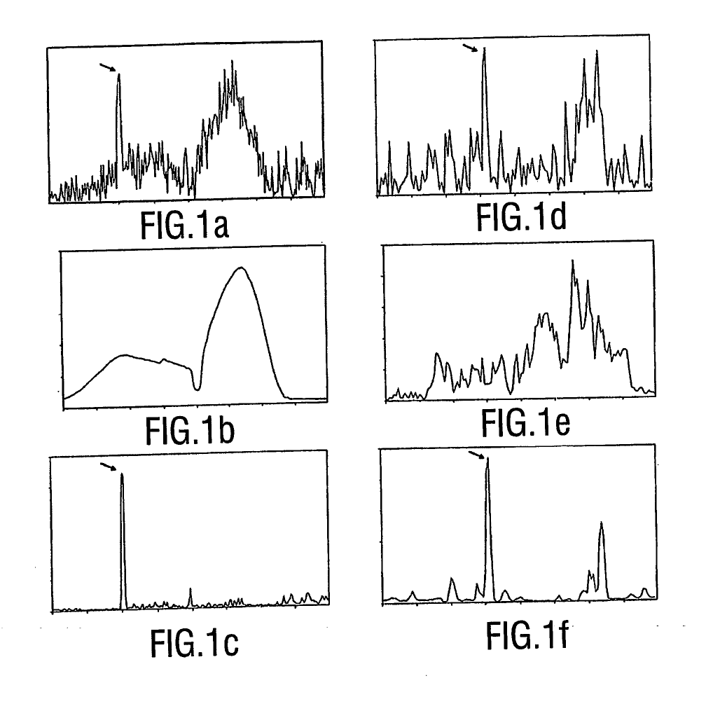

FIG. 1 shows the signal amplitudes plotted against the sampled values to illustrate the signal restoration using the expanded ME method in the event of strong interference of the input signals by transient artefacts.

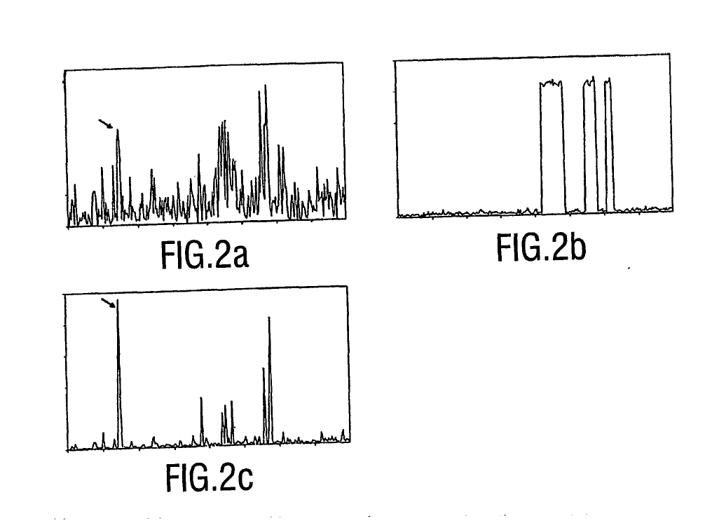

FIG. 2 shows the signal amplitudes plotted against the sampled values to illustrate the signal restoration using the expanded ME method in the event of strong interference of the input signals by image slice artefacts.

FIG. 1(a) shows an in vitro input signal having a total number of N=256 sampled values, in which the catheter position is marked by an arrow. The signal amplitudes on the ordinate are shown in graph form on the abscissa for the individual sampled values. The measurements were taken by means of a 1.5 Tesla MR tomography scanner (GyroScan ACS-NT, Philips Medical Systems) using a “spoiled” gradient-echo sequence (FOV=256 mm), where the catheter, which is an OptiMa catheter, has been placed in a tube phantom. The input signal is highly disrupted by transient artefacts, which are eliminated by forming and adapting a model function that is subtracted from the measured signals during the ME method. The model function used is the off-projection shown in (b), and this shows the recorded signals when the marking on the catheter is deactivated. The result after signal restoration has been completed is shown in (c), and the unambiguous determinability of the catheter position can be clearly seen here. The signal processing is associated with a considerable rise in the S/N and S/A ratios. Similarly, illustrations (d)-(f) show the signal restoration of an in vivo input signal which is highly disrupted by transient artefacts, where in this case the total number of sampled values was N=128. FIG. 1(d) in this instance shows the input signal, (e) shows the corresponding off-projection and (f) shows the result after signal restoration has been completed. The same method was used for the in vivo measurements as for the in vitro measurements, although in the case of the in vivo measurements an appropriate catheter was inserted into the aorta of a pig and a refocused gradient-echo sequence (FOV=300 mm) was used.

FIG. 2(a) shows a catheter signal with narrow image slice artefacts, where once again the signal amplitudes are shown in graph form for the individual sampled values and the position of the catheter is shown by an arrow. The model functions used within the context of the expanded ME method, which in the iterative method are again subtracted from measured signals, are shown in (b). In (c) it can be seen that after signal restoration the position of the catheter can be determined unambiguously, even though the artefacts occurring in (a) are very narrow and exceed the true catheter position in terms of amplitude.

1. A magnetic resonance method for locating interventional devices, in particular in vivo, in which the interventional device bears a marking which in the magnetic resonance acquisition influences the measured signals or generates its own measured signals, wherein the measured signals are processed by means of a one-dimensional signal processing method.

2. A method as claimed in claim 1, wherein the one-dimensional signal processing method is an iterative method.

3. A method as claimed in claim 2, wherein the iterative method is based on the maximum entropy method.

4. A method as claimed in claim 2, wherein, for artefacts occurring in the measured signals, model functions are formed, adapted and subtracted from the measured signals as the iterative method is carried out.

5. A method as claimed in claim 4, wherein the model functions are adapted to the recorded measured signals by way of a scaling parameter.

6. A method as claimed in claim 5, wherein the model functions are adapted anew to the recorded measured signals after each iteration step in the iterative method.

7. A method as claimed in claim 5, wherein the model functions are adapted to the recorded measured signals once, before the iterative method is carried out.

8. A method as claimed in claim 4, wherein the measured signals recorded when the marking on the interventional device is inactive are used as model function.

9. A method as claimed in claim 4, wherein rectangular or Gaussian functions are used as model functions.

10. A method as claimed in claim 4, the mean value of the difference between measured signal and model function is selected as start value for the iteration.

11. A method as claimed in claim 2, wherein the mean value of the measured signal is selected as start value for the iteration.

12. A method as claimed in claim 1, wherein high and/or low frequency signal fractions are eliminated in order to suppress noise and/or artefacts in the recorded measured signals.

13. A method as claimed in claim 1, wherein a filter with a finite or infinite impulse response is used as one-dimensional signal processing method.

14. A method as claimed in claim 13, wherein the filter is a Wiener filter or a bandpass filter.

15. A method as claimed in claim 1, wherein during the evaluation of a number of measured signals being used to locate the interventional device, after processing of the measured signals by means of the one-dimensional signal processing method a check as to coincidence of the positions of the interventional device determined by way of the processed measured signals is carried out.

16. A method as claimed in claim 1, wherein a number of measured signals being used to locate the interventional device are processed jointly in the one-dimensional signal processing method.

17. A method as claimed in claim 1, wherein the measured signals are recorded in parallel by a number of receiving coils.

18. A method as claimed in claim 1, wherein the one-dimensional signal processing method calculates the correlation of one or more measured signals.

19. An apparatus for locating interventional devices with the aid of magnetic resonance acquisition, in which the interventional device bears a marking which in the magnetic resonance acquisition influences the measured signals or generates its own measured signals, wherein the apparatus has program control for carrying out a method as claimed in claim 1.

20. A computer program for processing measured signals during the location of interventional devices with the aid of magnetic resonance acquisition, in which the interventional device bears a marking which in the magnetic resonance acquisition influences the measured signals or generates its own measured signals, wherein a method as claimed in claim 1 can be carried out by means of the computer program.
